(19) United States
(12) Patent Application Publication
(10) Pub. No.: US 2026/0219341 A1
(43) Pub. Date: Jul. 30, 2026

(54) SIGNAL TRANSMISSION CIRCUIT, OPTICAL COMMUNICATION MODULE, AND SYSTEM FOR MAGNETIC RESONANCE IMAGING

(71) Applicant: GE Precision Healthcare LLC, Waukesha, WI (US)

(72) Inventors: Yan Zhuang, Beijing (CN); Chen Zhang, Beijing (CN)

(21) Appl. No.: 19/456,285

(22) Filed: Jan. 22, 2026

(30) Foreign Application Priority Data
Jan. 26, 2025 (CN) 202510124949.0

(51) Int. Cl. G01R 33/36; H04B 10/50; H04B 10/69

(52) U.S. Cl. CPC G01R 33/3692; G01R 33/3607; G01R 33/3614; H04B 10/50; H04B 10/69

(57) ABSTRACT

A signal transmission circuit of a magnetic resonance imaging system, an optical communication module, and a system are provided. The signal transmission circuit includes: a processing circuit, which receives electrical signals from a plurality of signal receiving coil elements of the magnetic resonance imaging system, and respectively modulates the electrical signals from the plurality of signal receiving coil elements to corresponding non-overlapping frequency bands; and a receiving circuit, which is connected to the processing circuit and extracts the electrical signal in each frequency band from an output signal of the processing circuit. According to the present application, the number of channels of a signal is reduced, the difficulty of signal transmission and processing is reduced, and the number of elements of a signal transmission circuit is reduced. As a result, the difficulty of aspects such as chip design, manufacturing, and thermal management can be reduced.

### CROSS REFERENCE TO RELATED APPLICATIONS

The present application claims priority and benefit of Chinese Patent Application No. CN 202510124949.0 filed on January 26, 2025, which is incorporated herein by reference in its entirety.

### TECHNICAL FIELD

Embodiments of the present application relate to the technical field of medical devices, and in particular to a signal transmission circuit of a magnetic resonance imaging system, an optical communication module, and a system.

### BACKGROUND

Magnetic resonance (MR) imaging systems have been widely used in the field of medical diagnosis. A magnetic resonance imaging system generally has a main magnet, a gradient amplifier, a radio-frequency amplifier, a gradient coil, a transmitting chain module, a transmit/receive coil, a receiving chain module, an analog front end circuit, etc. The transmitting chain module generates a pulse signal and transmits the pulse signal that is amplified by the radio-frequency amplifier to the transmit/receive coil. The transmit/receive coil generates a radio-frequency excitation signal to excite a scanned subject to generate a magnetic resonance signal. After the excitation, by means of spatial encoding, the transmit/receive coil acquires the magnetic resonance signal. The resonance signal is transmitted by the receiving chain module, processed by the analog front end, and filled into a k- space, to reconstruct a medical image.

It should be noted that the above introduction of the background is only for the convenience of clearly and completely describing the technical solutions of the present application, and for the convenience of understanding for those skilled in the art.

### SUMMARY OF THEINVENTION

The miniaturization and integration of a circuit (e.g., a receiving chain module and an analog front end) represent a direction of development for magnetic resonance imaging systems. The inventors of the present application have found that a magnetic resonance signal received by the receiving chain module is a multi-channel (e.g., 32-channel) signal, and the analog front end circuit comprises a plurality of analog front end units. Each analog front end unit processes the magnetic resonance signal of a corresponding channel. Therefore, during the integrated design of the analog front end, the plurality of analog front end units need to be integrated onto one chip (e.g., an application-specific integrated circuit (ASIC)), which poses numerous challenges to aspects such as chip design, manufacturing, and thermal management. Therefore, a technical problem to be solved is how to reduce the scale of a circuit while maintaining circuit functionality, thereby reducing the difficulty of aspects such as chip design, manufacturing, and thermal management.

To address the foregoing technical problem or at least similar technical problems, embodiments of the present application provide a signal transmission circuit of a magnetic resonance imaging system, an optical communication module, and a system. The signal transmission circuit respectively loads electrical signals from a plurality of signal receiving coils of the magnetic resonance imaging system onto corresponding frequency bands and transmits the electrical signals to a receiving circuit. The receiving circuit extracts the corresponding signals from the different frequency bands. Therefore, the number of channels of a signal is reduced, the difficulty of signal transmission and processing is reduced, and the number of elements of the signal transmission circuit is reduced. Thus, the difficulty of aspects such as chip design, manufacturing, and thermal management can be reduced.

According to an aspect of the embodiments of the present application, a signal transmission circuit of a magnetic resonance imaging system is provided. The signal transmission circuit comprises: a processing circuit, which receives electrical signals from a plurality of signal receiving coil elements of the magnetic resonance imaging system, and respectively modulates the electrical signals from the plurality of signal receiving coil elements to corresponding non-overlapping frequency bands. Further, the signal transmission circuit includes a receiving circuit, which is connected to the processing circuit and extracts the electrical signal in each frequency band from an output signal of the processing circuit.

According to an aspect of the embodiments of the present application, an optical communication module of a magnetic resonance imaging system is provided. The optical communication module comprises: a plurality of electro-optical converters, each electro-optical converter being separately connected to one of a plurality of signal receiving coil elements of the magnetic resonance imaging system and converting an electrical signal received by the signal receiving coil element into an optical signal. The optical communication module also includes an optical switch, an input end of the optical switch being connected to the plurality of electro- optical converters, an output end of the optical switch being connected to an optical fiber, and the optical switch guiding the optical signal generated by each electro-optical converter to a corresponding transmission channel. Further, the optical communication module includes the optical fiber, one end of the optical fiber being connected to the output end of the optical switch, and the optical fiber transmitting the optical signal in the transmission channel; and a photoelectric converter, which is connected to the optical fiber, and converts the optical signal received from the optical fiber into an electrical signal and outputs the electrical signal to a processing circuit of the magnetic resonance imaging system.

One of the beneficial effects of the embodiments of the present application is that: The signal transmission circuit loads electrical signals from a plurality of signal receiving coils of the magnetic resonance imaging system onto corresponding frequency bands and transmits the electrical signals to a receiving circuit. The receiving circuit extracts the corresponding signals from the different frequency bands. Therefore, the number of channels of a signal is reduced, the difficulty of signal transmission and processing is reduced, and the number of elements of the signal transmission circuit is reduced. As a result, the difficulty of aspects such as chip design, manufacturing, and thermal management can be reduced.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a magnetic resonance imaging system according to an embodiment of the present application;

FIG. 2 is a schematic diagram of a signal transmission circuit of a magnetic resonance imaging system according to an embodiment of the present application;

FIG. 3 is a schematic diagram of processing, by a processing circuit, electrical signals of a plurality of channels to obtain an output signal Vout;

FIG. 4 is another schematic diagram of a signal transmission circuit of a magnetic resonance imaging system according to an embodiment of the present application;

FIG. 5 is a schematic diagram of an optical communication module according to an embodiment of the present application; and

FIG. 6 is a schematic diagram of the composition of a magnetic resonance imaging system according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms "first", "second", etc., are used to distinguish different elements, but do not represent a spatial arrangement or temporal order, etc., of these elements, and these elements should not be limited by these terms. The term "and/or" includes any and all combinations of one or more associated listed terms. The terms "comprise", "include", "have", etc., refer to the presence of described features, elements, components, or assemblies, but do not exclude the presence or addition of one or more other features, elements, components, or assemblies.

In the embodiments of the present application, the singular forms "a" and "the" include the plural forms, and should be broadly construed as "a type of' or "a class of' rather than being limited to the meaning of "one". In addition, the term "the" should be construed as including both the singular and plural forms, unless otherwise specified in the context. In addition, the term "according to" should be construed as "at least in part according to..." and the term "based on" should be construed as "at least in part based on...", unless otherwise explicitly specified in the context.

In the embodiments of the present application, the term "key point" may be equivalently replaced with "key coordinate point", "landmark", "landmark point", or the like. The term "subject" may be equivalently replaced with "examination subject", "examined subject", "scanned subject", "subject to be scanned", "patient", etc., which may be a person, an animal, or other objects, etc. The term "object" may be equivalently replaced with "detection object", "detected object", or "research subject", etc., which may be a part or other components, etc.

In the embodiments of the present application, the term "include/comprise" when used herein refers to the presence of features, integrated components, steps, or assemblies, but does not preclude the presence or addition of one or more other features, integrated components, steps, or assemblies.

The features described and/or illustrated for one implementation may be used in one or more other implementations in the same or similar way, be combined with features in other implementations, or replace features in other implementations.

In the embodiments of the present application, a signal transmission circuit and an optical communication module are applicable to a variety of medical imaging scenarios, including, but not limited to, magnetic resonance imaging (MRI), computed tomography (CT), ultrasound imaging, positron emission computed tomography (PET), single photon emission computed tomography (SPECT), PET/CT, PET/MR, or any other suitable medical imaging scenarios.

In the embodiments of the present application, the present application is exemplarily described by using a magnetic resonance imaging (MRI) scenario as an example. It should be understood that the contents of the embodiments of the present application are also applicable to other medical imaging scenarios.

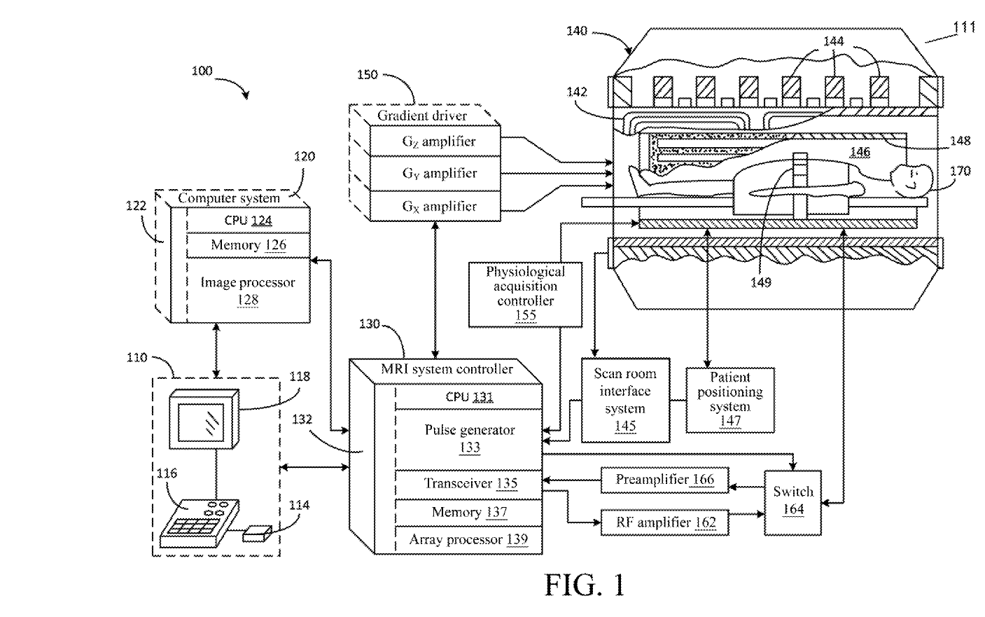

For ease of understanding, FIG. 1 is a schematic diagram of a magnetic resonance imaging (MRI) system 100 according to an embodiment of the present application.

The MRI system 100 includes a scanning unit 111. The scanning unit 111 is used to perform a magnetic resonance scan of a subject (e.g., a human body) 170 to generate image data of a region of interest of the subject 170, wherein the region of interest may be a pre-determined anatomical site or anatomical tissue.

The operation of the MRI system 100 is controlled by an operator workstation 110. The operator workstation 110 includes an input device 114, a control panel 116, and a display 118. The input device 114 may be a joystick, a keyboard, a mouse, a trackball, a touch- activated screen, voice control, or any similar or equivalent input device. The control panel 116 may include a keyboard, a touch-activated screen, voice control, a button, a slider, or any similar or equivalent control device. The operator workstation 110 is coupled to and communicates with a computer system 120 that enables an operator to control the generation and display of images on the display 118. The computer system 120 includes various components that communicate with one another via an electrical and/or data connection module 122. The connection module 122 may employ a direct wired connection, a fiber optic connection, a wireless communication link, etc. The computer system 120 may include a central processing unit (CPU) 124, a memory 126, and an image processor 128. In some embodiments, the image processor 128 may be replaced by medical imaging functions implemented in the CPU 124. The computer system 120 may be connected to an archive media device, a persistent or backup memory, or a network. The computer system 120 may be coupled to and communicate with a separate MRI system controller 130.

The MRI system controller 130 includes a set of components that communicate with one another via an electrical and/or data connection module 132. The connection module 132 may employ a direct wired connection, a fiber optic connection, a wireless communication link, etc. The MRI system controller 130 may include a CPU 131, a sequence pulse generator (also known as a pulse generator) 133 that communicates with the operator workstation 110, a transceiver (also known as an RF transceiver) 135, a memory 137, and an array processor 139.

In some embodiments, the sequence pulse generator 133 may be integrated into a resonance assembly 140 of the scanning unit 111 of the MRI system 100. The MRI system controller 130 may receive a command from the operator workstation 110, and is coupled to the scanning unit 111 to indicate an MRI scanning sequence to be executed during an MRI scan, so as to be used to control the scanning unit 111 to execute the flow of the aforementioned magnetic resonance scan. The MRI system controller 130 is further coupled to a gradient driver system (also known as gradient driver) 150 and communicates therewith, and the gradient driver system is coupled to a gradient coil assembly 142 to generate a magnetic field gradient during an MRI scan.

The sequence pulse generator 133 may further receive data from a physiological acquisition controller 155 that receives signals from a plurality of different sensors (e.g., electrocardiogram (ECG) signals from electrodes attached to a patient, etc.), the sensors being connected to a subject or patient 170 undergoing an MRI scan. The sequence pulse generator 133 is coupled to and communicates with a scan room interface system 145 that receives signals from various sensors associated with the state of the resonance assembly 140. The scan room interface system 145 is further coupled to and communicates with a patient positioning system 147 that sends and receives signals to control a patient table to move to a desired position for an MRI scan.

The MRI system controller 130 provides gradient waveforms to the gradient driver system 150, and the gradient driver system includes Gx (x direction), Gy (y direction), and Gz (z direction) amplifiers, etc. Each of the Gx, Gy, and Gz gradient amplifiers excites a corresponding gradient coil in the gradient coil assembly 142, so as to generate a magnetic field gradient used to spatially encode an MR signal during an MRI scan. The gradient coil assembly 142 is disposed within the resonance assembly 140, and the resonance assembly further includes a superconducting magnet having a superconducting coil 144 that, in operation, provides a static uniform longitudinal magnetic field Bo throughout a cylindrical imaging volume 146. The resonance assembly 140 further includes an RF body coil 148, which, in operation, provides a transverse magnetic field B1, the transverse magnetic field B1 being substantially perpendicular to Bo throughout the entire cylindrical imaging volume 146. The resonance assembly 140 may further include an RF surface coil 149 for imaging different anatomical structures of a patient undergoing an MRI scan. The RF body coil 148 and the RF surface coil 149 may be configured to operate in a transmit and receive mode, a transmit mode, or a receive mode.

The x direction may also be referred to as a frequency encoding direction or a kx direction in the k-space, the y direction may be referred to as a phase encoding direction or a ky direction in the k-space, and the z direction may be referred to as a layer surface selection (layer selection) direction. Gx may be used for frequency encoding or signal readout, and is generally referred to as a frequency encoding gradient or a readout gradient. Gy may be used for phase encoding, and is generally referred to as a phase encoding gradient. Gz may be used for slice (layer) position selection to obtain k-space data. It should be noted that a layer selection direction, a phase encoding direction, and a frequency encoding direction may be modified according to actual requirements.

The subject or patient 170 of the MRI scan may be positioned within the cylindrical imaging volume 146 of the resonance assembly 140. The transceiver 135 in the MRI system controller 130 generates RF excitation pulses amplified by an RF amplifier 162, and provides the same to the RF body coil 148 by means of a transmit/receive switch (also known as a T/R switch or a switch) 164.

As described above, the RF body coil 148 and the RF surface coil 149 may be used to transmit RF excitation pulses and/or receive obtained MR signals from a patient undergoing an MRI scan. The MR signals emitted by excited nuclei in the patient of the MRI scan may be sensed and received by the RF body coil 148 or the RF surface coil 149 and sent back to a preamplifier 166 by means of the T/R switch 164. The T/R switch 164 may be controlled by a signal from the sequence pulse generator 133 to electrically connect the RF amplifier 162 to the RF body coil 148 in the transmit mode and to connect the preamplifier 166 to the RF body coil 148 in the receive mode. The T/R switch 164 may further enable the RF surface coil 149 to be used in the transmit mode or the receive mode.

In some embodiments, the MR signals sensed and received by the RF body coil 148 or the RF surface coil 149 and amplified by the preamplifier 166 are stored in the memory 137 for post-processing as a raw k-space data array. A reconstructed magnetic resonance image may be obtained by transforming/processing the stored raw k-space data.

In some embodiments, the MR signals sensed and received by the RF body coil 148 or the RF surface coil 149 and amplified by the preamplifier 166 are demodulated, filtered, and digitized in a receiving portion of the transceiver 135, and transmitted to the memory 137 in the MRI system controller 130. For each image to be reconstructed, the data is rearranged into separate k-space data arrays, each of these separate k-space data arrays being inputted into the array processor 139, and the array processor being operated to transform the data into an array of image data by Fourier transform.

The array processor 139 uses transform methods, most commonly Fourier transform, to create images from received MR signals. These images are transmitted to the computer system 120 and stored in the memory 126. In response to commands received from the operator workstation 110, the image data may be stored in a long-term memory, or may be further processed by the image processor 128 and transmitted to the operator workstation 110 for presentation on the display 118.

In various embodiments, components of the computer system 120 and the MRI system controller 130 may be implemented on the same computer system or on a plurality of computer systems. It should be understood that the MRI system 100 shown in FIG. 1 is intended for illustration. Suitable MRI systems may include more, fewer, and/or different components.

The MRI system controller 130 and the image processor 128 may separately or collectively include a computer processor and a storage medium. The storage medium records a predetermined data processing program to be executed by the computer processor. For example, the storage medium may store a program used to implement scanning processing (such as a scan flow and an imaging sequence), image reconstruction, medical imaging, etc. For example, the storage medium may store a computer program for determining an orientation of a subject according to the embodiments of the present invention. The described storage medium may include, for example, a ROM, a floppy disk, a hard disk, an optical disk, a magneto-optical disk, a CD-ROM, or a non-volatile memory card.

Embodiments of the present application further provide a signal transmission circuit of a magnetic resonance imaging system.

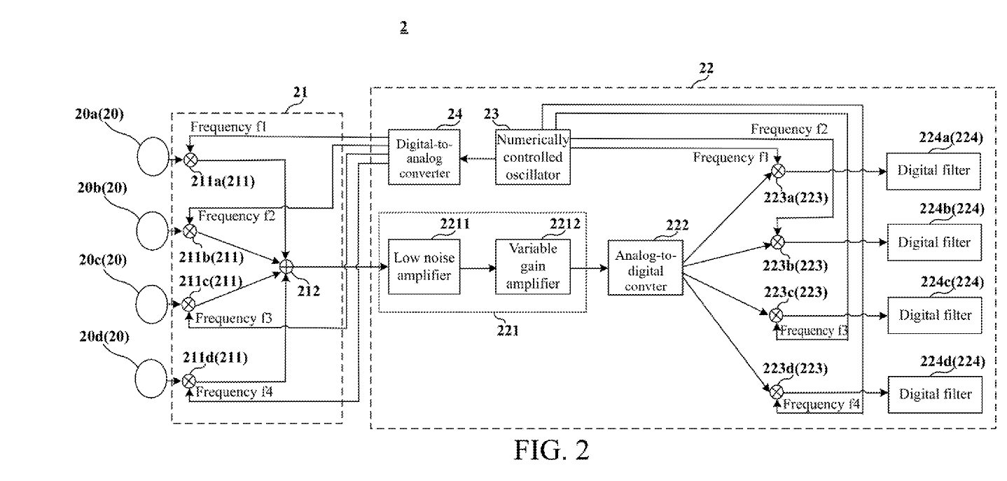

FIG. 2 is a schematic diagram of a signal transmission circuit of a magnetic resonance imaging system according to an embodiment of the present application. As shown in FIG. 2, a signal transmission circuit 2 includes: a processing circuit 21 and a receiving circuit 22.

The processing circuit 21 receives electrical signals from a plurality of signal receiving coil elements 20 of a magnetic resonance imaging system, and respectively modulates the electrical signals from the plurality of signal receiving coil elements 20 to corresponding non-overlapping frequency bands for output. The receiving circuit 22 is connected to the processing circuit 21. The receiving circuit 22 extracts the electrical signal in each frequency band from an output signal of the processing circuit 21.

In the present application, the magnetic resonance imaging system is, for example, the magnetic resonance imaging (MRI) system 100 shown in FIG. 1. The signal receiving coil elements 20 may receive magnetic resonance (MR) signals from a patient undergoing MRI scanning. To be specific, the electrical signals from the signal receiving coil elements 20 are the MR signals received by the signal receiving coil elements 20, wherein the electrical signals from the signal receiving coil elements 20 may be analog signals. The magnetic resonance imaging system 100 may have a plurality of signal receiving coil elements 20, wherein each signal receiving coil element 20 may correspond to one signal channel. For example, four signal receiving coil elements 20 (i.e., 20a, 20b, 20c, and 20d) shown in FIG. 2 may correspond to channel 1, channel 2, channel 3, and channel 4, respectively.

The four signal receiving coil elements 20 or four signal channels shown in FIG. 2 are merely examples, and the present application is not limited thereto. For example, the number of signal receiving coil elements 20 or signal channels in the magnetic resonance imaging system 100 may be 8, 16, 32, or another number, with no specific numerical limitation imposed by the present application. The following description of the present application will use four signal receiving coil elements 20 as an example, and such description is equally applicable to cases in which the number of signal receiving coil elements 20 is other values.

In the present application, the plurality of signal receiving coil elements 20 may be components of at least one of the radio-frequency (RF) body coil 148 and the RF surface coil 149 shown in FIG. 1. For an explanation of the operating principle of each receiving coil element 20, reference may be made to the description of the RF body coil 148 or the RF surface coil 149 operating in a receive mode.

As shown in FIG. 2, in some embodiments, the processing circuit 21 includes: mixers 211 and a synthesizer 212.

There are a plurality of mixers 211. In some examples, the number of mixers 211 is the same as the number of signal receiving coil elements 20. For example, as shown in FIG. 2, there are four mixers 211, namely mixers 211 a, 211b, 211c, and 211d. The plurality of mixers respectively 211 correspond to the plurality of signal receiving coil elements 20. For example, the mixers 211a, 211b, 211c, and 211d correspond to the signal receiving coil elements 20a, 20b, 20c, and 20d, respectively.

Each mixer 211 mixes an electrical signal (e.g., the electrical signal is an analog signal) with a carrier signal (e.g., the carrier signal is an analog signal) to implement signal modulation, so as to modulate the electrical signal of each channel to a corresponding frequency band, achieving frequency spectrum shifting of the original electrical signal in the frequency domain. Each carrier signal may be a tone signal (i.e., a signal with a single frequency), and frequencies of respective carrier waves are different from each other such that the frequency bands of the modulated signals do not overlap with one another. In addition, each carrier signal may alternatively be referred to as a local oscillator signal.

For example, the mixer 211a mixes an electrical signal Si from the signal receiving coil element 20a (i.e., an electrical signal of channel 1) with a carrier signal C1 having a frequency of fl to obtain a modulated signal M1 with a frequency band of W1. The mixer 211b mixes an electrical signal S2 from the signal receiving coil element 20b (i.e., an electrical signal of channel 2) with a carrier signal C2 having a frequency of f2 to obtain a modulated signal M2 with a frequency band of W2. The mixer 211c mixes an electrical signal S3 from the signal receiving coil element 20c (i.e., an electrical signal of channel 3) with a carrier signal C3 having a frequency of f3 to obtain a modulated signal M3 with a frequency band of W3. The mixer 211d mixes an electrical signal S4 from the signal receiving coil element 20d (i.e., an electrical signal of channel 4) with a carrier signal C4 having a frequency of f4 to obtain a modulated signal M4 with a frequency band of W4. Any two of the frequency bands W1, W2, W3, and W4 do not overlap.

The synthesizer 212 synthesizes the signals generated by the plurality of mixers 211 to form the output signal of the processing circuit 21. For example, the synthesizer 212 synthesizes the modulated signals M1, M2, M3, and M4 to form an output signal Vout of the processing circuit 21.

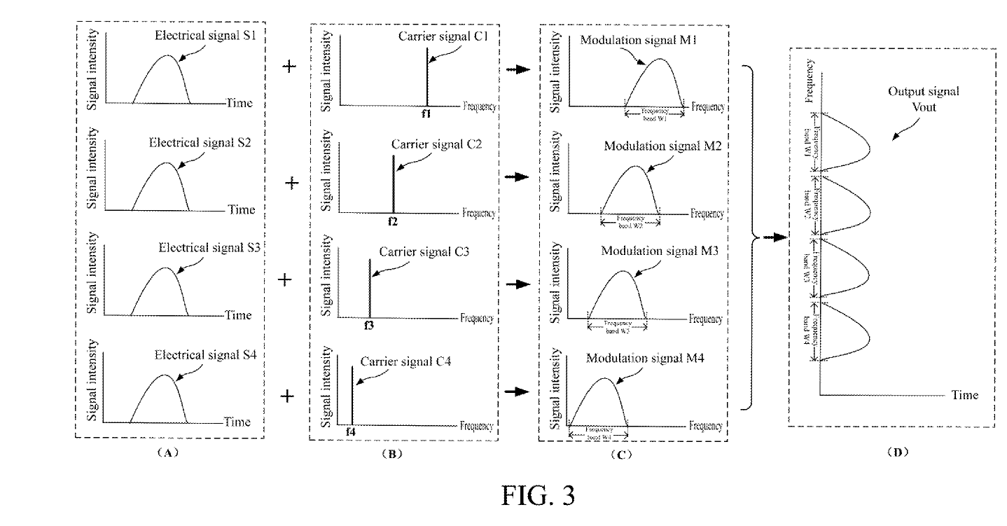

FIG. 3 is a schematic diagram of processing, by a processing circuit, electrical signals of a plurality of channels to obtain an output signal Vout.

FIG. 3(A) shows time-domain waveforms of the electrical signals S1 S2, S3, and S4 from the plurality of signal receiving coil elements 20, wherein the vertical axis represents signal intensity and the horizontal axis represents time.

FIG. 3(B) shows frequency-domain waveforms of the carrier signals C1, C2, C3, and C4 used by the mixers 211. The frequency bands of the respective carrier signals are W1, W2, W3, and W4, and the frequency bands do not overlap with one another. The vertical axis represents signal intensity and the horizontal axis represents frequency.

FIG. 3(C) shows frequency-domain waveforms of the modulated signals M1, M2, M3, and M4 generated by the mixers 211, wherein the vertical axis represents signal intensity and the horizontal axis represents frequency.

FIG. 3(D) shows a time-frequency-domain waveform of the output signal Vout generated by the synthesizer 212 after synthesizing the modulated signals M1, M2, M3, and M4, wherein the vertical axis represents frequency and the horizontal axis represents time.

The processing circuit 21 of the present application modulates the electrical signals from the plurality of signal receiving coil elements 20 (i.e., the electrical signals of the plurality of channels) onto different frequency bands to generate the output signal Vout. The output signal Vout may be transmitted by means of a single transmission channel, thereby reducing a plurality of transmission lines originally required for transmitting the electrical signals of the plurality of channels (e.g., one transmission line is required for the electrical signal of each channel, so that four transmission lines would be required for transmitting electrical signals of four channels) to one. Therefore, the number of signal transmission channels is reduced.

As shown in FIG. 2, the receiving circuit 22 includes: an amplifying circuit 221, an analog-to-digital converter (ADC) 222, multipliers 223, and digital filters 224.

The amplifying circuit 221 receives the output signal Vout from the processing circuit 21 and amplifies the output signal Vout. In some examples, the amplifying circuit 221 may be an analog amplifying circuit. For example, the amplifying circuit 221 may include at least one of a low noise amplifier (LNA) 2211 and a variable gain amplifier (VGA) 2212.

In the example shown in FIG. 2, the amplifying circuit 221 includes both the low noise amplifier 2211 and the variable gain amplifier (VGA) 2212. The low noise amplifier 2211 receives the output signal Vout and performs low noise amplification on the output signal Vout. The variable gain amplifier 2212 further amplifies the signal amplified by the low noise amplifier 2211.

The analog-to-digital converter (ADC) 222 converts the signal amplified by the amplifying circuit 221 into a digital signal. For example, the analog-to-digital converter 222 is connected to the variable gain amplifier 2212.

There are a plurality of multipliers 223. In some examples, the number of multipliers 223 may be the same as the number of mixers 211. For example, as shown in FIG. 2, there are four multipliers 223, namely multipliers 223 a, 223b, 223c, and 223d.

The multipliers 223 respectively multiply a plurality of carrier signals (e.g., carrier signals in the form of digital signals) with the digital signal outputted by the analog-to-digital converter 222, thereby extracting the electrical signal in each frequency band from the digital signal. The extracted electrical signal is in the form of a digital signal. The frequency bands of the carrier signals do not overlap with one another.

For example, the multiplier 223a multiplies a carrier signal Cla with a frequency of f1 by the digital signal outputted by the analog-to-digital converter 222 to obtain an electrical signal Sla. The carrier signal Cla has the same frequency as the aforementioned carrier signal C1. The carrier signal Cla is a signal in a digital form (i.e., a digital signal), while the carrier signal C1 is an analog signal. The electrical signal Sla corresponds to the aforementioned electrical signal Si. The electrical signal Sla is a signal in a digital form (i.e., a digital signal), while the electrical signal S1 is an analog signal.

The multiplier 223b multiplies a carrier signal C2a with a frequency of f2 by the digital signal outputted by the analog-to-digital converter 222 to obtain an electrical signal S2a. The carrier signal C2a has the same frequency f2 as the aforementioned carrier signal C2. The carrier signal C2a is a signal in a digital form (i.e., a digital signal), while the carrier signal C2 is an analog signal. The electrical signal S2a corresponds to the aforementioned electrical signal S2. The electrical signal S2a is a signal in a digital form (i.e., a digital signal), while the electrical signal S2 is an analog signal.

The multiplier 223c multiplies a carrier signal C3a with a frequency of f3 by the digital signal outputted by the analog-to-digital converter 222 to obtain an electrical signal S3a. The carrier signal C3a has the same frequency as the aforementioned carrier signal C3. The carrier signal C3a is a signal in a digital form (i.e., a digital signal), while the carrier signal C3 is an analog signal. The electrical signal S3a corresponds to the aforementioned electrical signal S3. The electrical signal S3a is a signal in a digital form (i.e., a digital signal), while the electrical signal S3 is an analog signal.

The multiplier 223d multiplies a carrier signal C4a with a frequency of f4 by the digital signal outputted by the analog-to-digital converter 222 to obtain an electrical signal S4a. The carrier signal C4a has the same frequency as the aforementioned carrier signal C4. The carrier signal C4a is a signal in a digital form (i.e., a digital signal), while the carrier signal C4 is an analog signal. The electrical signal S4a corresponds to the aforementioned electrical signal S4. The electrical signal S4a is a signal in a digital form (i.e., a digital signal), while the electrical signal S4 is an analog signal.

The electrical signals in a digital form generated by the plurality of multipliers 223 (e.g., electrical signals S1a, S2a, S3a, and S4a) respectively correspond to the plurality of channels (e.g., channel 1, channel 2, channel 3, and channel 4).

In the present application, there are a plurality of digital filters 224. In some examples, the number of digital filters 224 may be the same as the number of mixers 223. For example, as shown in FIG. 2, there are four digital filters 224, namely digital filters 224a, 224b, 224c, and 224d.

The digital filters 224 respectively filter the signals (e.g., electrical signals S1a, S2a, S3a, and S4a) generated by the corresponding multipliers 223. In some examples, each digital filter 224 may be a band-pass filter, and a frequency band for filtering may correspond to the frequency band of the carrier signal used by the corresponding multiplier 223. For example, the frequency bands filtered by the digital filters 224a, 224b, 224c, and 224d are W1, W2, W3, and W4, respectively. The digital filters 224 may filter out signals outside the corresponding frequency bands and obtain signals within the corresponding frequency bands.

As shown in FIG. 2, in some embodiments, the signal transmission circuit 2 further includes: a numerically controlled oscillator (NCO) 23 and a digital-to-analog converter (DAC) 24.

The numerically controlled oscillator 23 generates a plurality of carrier signals in the form of digital signals. The plurality of carrier signals in the form of digital signals may be transmitted to the plurality of multipliers 223. For example, the numerically controlled oscillator 23 may be connected to the plurality of multipliers 223. The numerically controlled oscillator 23 generates the foregoing carrier signals C1a, C2a, C3a, and C4a in the form of digital signals. The carrier signals C1a, C2a, C3a, and C4a in the form of digital signals are transmitted to the multipliers 223 a, 223b, 223c, and 223d, respectively.

The digital-to-analog converter 24 is connected to the numerically controlled oscillator 23 and the plurality of mixers 211. The digital-to-analog converter 24 converts the plurality of carrier signals in the form of digital signals (e.g., carrier signals C1a, C2a, C3a, and C4a) generated by the numerically controlled oscillator 23 into a plurality of carrier signals in the form of analog signals (e.g., carrier signals C1, C2, C3, and C4), and transmits the carrier signals in the form of analog signals to the plurality of mixers 211. For example, the carrier signals C1, C2, C3, and C4 in the form of analog signals are transmitted to the mixers 211a, 221b, 221c, and 221d, respectively.

In the present application, the numerically controlled oscillator 23, the digital-to- analog converter 24, and the receiving circuit 22 are integrated into the same chip, thereby improving the integration level of the circuit. For example, the same chip is an application- specific integrated circuit (ASIC) chip.

In the present application, as shown in FIG. 2, the processing circuit 21 may load the electrical signals of the plurality of channels onto different frequency bands to form the output signal Vout, and transmit the output signal Vout to the receiving circuit 22 by means of one transmission channel, thereby simplifying the transmission channel wiring. Furthermore, the receiving circuit 22 is provided with one amplifying circuit 221 and one analog-to-digital converter 222 to perform amplification and analog-to-digital conversion on the output signal Vout. Therefore, there is no need to provide a plurality of amplifying circuits and a plurality of analog-to-digital converters for separately performing amplification and analog-to-digital conversion on the electrical signals of the plurality of channels. The hardware of the circuit is simplified, and the difficulty of aspects such as chip design, manufacturing, and thermal management is also reduced.

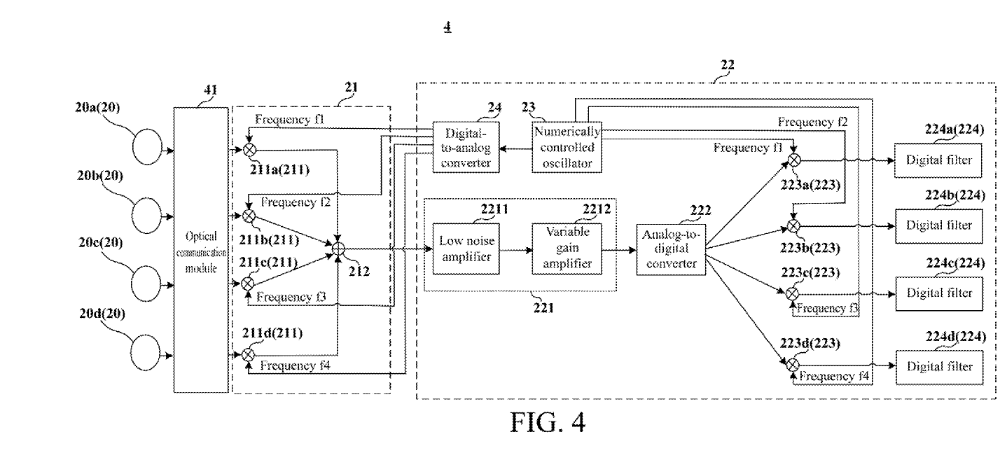

FIG. 4 is another schematic diagram of a signal transmission circuit of a magnetic resonance imaging system according to an embodiment of the present application. As shown in FIG. 4, a signal transmission circuit 4 differs from the signal transmission circuit 2 in FIG. 2 in that the signal transmission circuit 4 in FIG. 4 further includes an optical communication module 41 in addition to all the components of the signal transmission circuit 2 in FIG. 2. The same components in FIG. 4 and FIG. 2 are denoted by the same reference numerals, and for the description of the same components, reference may be made to the relevant description of FIG. 2.

The following describes the difference between FIG. 4 and FIG. 2.

As shown in FIG. 4, the optical communication module 41 is connected between the plurality of signal receiving coil elements 20 and the processing circuit 21.

The optical communication module 41 may convert electrical signals (e.g., analog signals) received by the plurality of signal receiving coil elements 20 into optical signals, transmit the optical signals to a side of the processing circuit 21, and convert the optical signals transmitted to the side of the processing circuit 21 into electrical signals (e.g., analog signals) to be outputted to the processing circuit 21. Therefore, in a transmission path for transferring the electrical signals (e.g., analog signals) received by the plurality of signal receiving coil elements 20 to the processing circuit 21, replacing wire links such as cables (e.g., coaxial cables for transmitting radio-frequency signals) with an optical communication link can reduce signal loss, improve signal quality, and make the wiring of the optical communication link simpler and more flexible. In contrast, the wire links incur certain signal loss when transmitting the radio- frequency signals, and the wire links have high requirements for electromagnetic shielding, resulting in complex wiring.

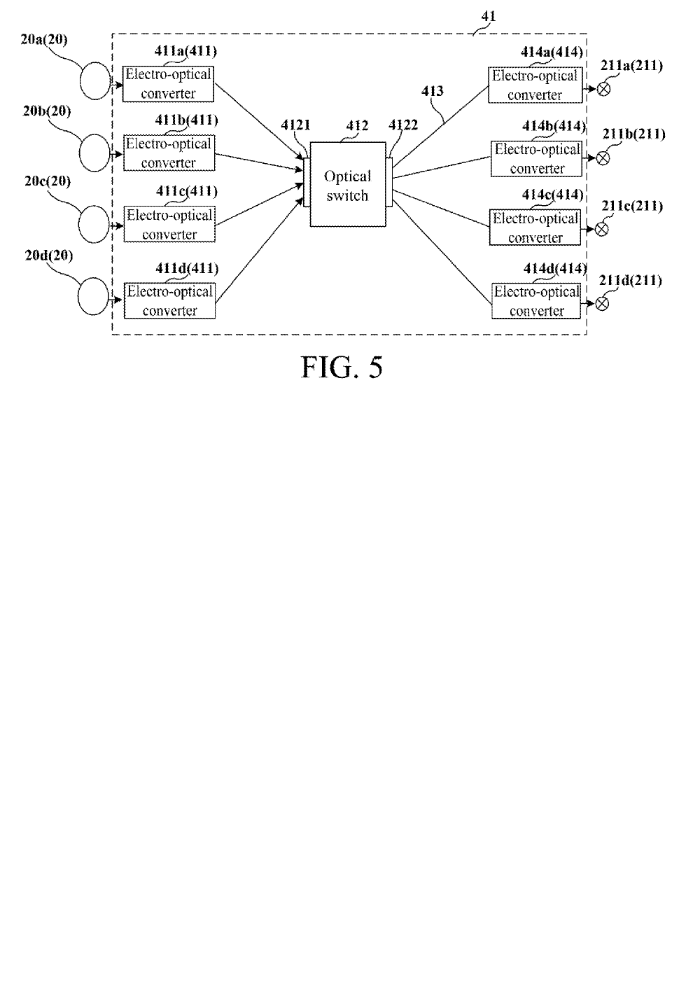

FIG. 5 is a schematic diagram of an optical communication module. As shown in FIG. 5, the optical communication module 41 includes: electro-optical converters 411, an optical switch 412, an optical fiber 413, and a photoelectric converter 414.

There are a plurality of electro-optical converters 411. In some examples, the number of electro-optical converters 411 is the same as the number of signal receiving coil elements 20. For example, as shown in FIG. 5, there are four electro-optical converters 411, namely electro-optical converters 411a, 411b, 411c, and 411d.

Each electro-optical converter 411 is separately connected to the corresponding signal receiving coil element 20, and converts the electrical signal received by the signal receiving coil element 20 into an optical signal. For example, the electro-optical converters 411a, 411b, 411c, and 411d are connected to the signal receiving coil elements 20a, 20b, 20c, and 20d, respectively, and convert the electrical signals in the signal receiving coil elements 20a, 20b, 20c, and 20d into optical signals.

In some examples, each electro-optical converter 411 may, based on radio-frequency over fiber (RFoF) technology, modulate an optical wave using a radio-frequency electrical signal received by the signal receiving coil element 20, thereby converting the radio-frequency electrical signal into an optical signal.

An input end 4121 of the optical switch 412 is connected to the plurality of electro- optical converters 411 (e.g., via optical fibers). The optical switch 412 guides the optical signal outputted by each electro-optical converter 411 to a corresponding transmission channel (e.g., an optical transmission channel). For example, the number of input ends 4121 of the optical switch 412 is M (wherein M is a natural number), for receiving optical signals from M electro-optical converters 411. The number of output ends 4122 of the optical switch 412 is N (wherein N is a natural number), for outputting optical signals to N transmission channels. The optical switch 412 is internally provided with optical path switching elements for selecting the optical transmission channels connecting the input ends 4121 and the output ends 4122. .

One end of the optical fiber 413 is connected to the output end 4122 of the optical switch 412. The optical fiber 413 transmits the optical signal outputted from the output end 4122. There may be one or more optical fibers 413. For example, the number of optical fibers 413 may be less than or equal to the number of output ends 4122 of the optical switch 412.

The photoelectric converter 414 is connected to the other end of the optical fiber 413, converts the optical signal received from the optical fiber 413 into an electrical signal, and outputs the electrical signal to the processing circuit 21. There may be one or more photoelectric converters 414. In some examples, the number of photoelectric converters 414 is equal to the number of optical fibers 413 and also equal to the number of mixers 211 in the processing circuit 21. For example, the photoelectric converters 414a, 414b, 414c, and 414d are connected to the multipliers 211a, 211b, 211c, and 211d, respectively.

In addition, in the present application, the optical communication module 41 may not be combined with the signal transmission circuit of FIG. 2. For example, the photoelectric converter 414 of the optical communication module 41 may not be connected to the processing circuit 21, but instead to other processing circuits of the magnetic resonance imaging system 100, thereby converting the optical signal into an electrical signal and transmitting the electrical signal to the other processing circuits. For example, the other processing circuits may be the preamplifier 166 shown in FIG. 1.

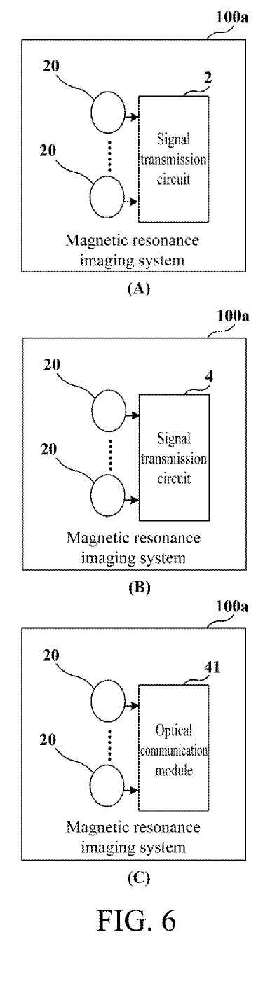

Embodiments of the present application further provide a magnetic resonance imaging (MRI) system. FIG. 6 is a schematic diagram of the composition of the magnetic resonance imaging system. As shown in FIGS. 6(A), 6(B), and 6(C), a magnetic resonance imaging system 100a may include a plurality of signal receiving coil elements 20, and the magnetic resonance imaging system 100a may further include the signal transmission circuit 2 (as shown in FIG. 6(A)), the signal transmission circuit 4 (as shown in FIG. 6(B)), or the optical communication module 41 (as shown in FIG. 6(C)) described in the aforementioned embodiments. The signal transmission circuit 2, the signal transmission circuit 4, or the optical communication module 41 is connected to the plurality of signal receiving coil elements 20.

The specific compositional structure of the magnetic resonance imaging system 100a in FIG. 6 may be similar to that of the magnetic resonance imaging system 100 in FIG. 1.

In the magnetic resonance imaging system 100a shown in FIGS. 6(A), 6(B), and 6(C), the plurality of signal receiving coil elements 20 may be components of at least one of the radio-frequency (RF) body coil 148 and the RF surface coil 149 shown in FIG. 1.

In FIGS. 6(A) or 6(B), the signal transmission circuit 2 or the signal transmission circuit 4 of the magnetic resonance imaging system 100a may replace the preamplifier 166 of the magnetic resonance imaging system 100 in FIG. 1.

In FIG. 6(C), the optical communication module 41 of the magnetic resonance imaging system 100a may be connected to the preamplifier 166 of the magnetic resonance imaging system 100 in FIG. 1. Therefore, replacing wire links such as cables (e.g., coaxial cables for transmitting radio-frequency signals) with an optical communication link to transmit the electrical signals (e.g., analog signals) received by the signal receiving coil elements 20 can reduce signal loss and improve signal quality. In addition, the optical communication link has low requirements for electromagnetic shielding, enabling more flexible wiring in the magnetic resonance imaging system.

The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, the software modules can be stored in the MEGA-SIM card or the large-capacity flash memory apparatus.

One or more of the functional blocks and/or one or more combinations of the functional blocks shown in the accompanying drawings may be implemented as a general- purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for executing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific implementations. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

What is claimed is:

1. A signal transmission circuit of a magnetic resonance imaging system, characterized by comprising:a processing circuit, which receives electrical signals from a plurality of signal receiving coil elements of the magnetic resonance imaging system, and respectively modulates the electrical signals from the plurality of signal receiving coil elements to corresponding non- overlapping frequency bands; anda receiving circuit, which is connected to the processing circuit and extracts the electrical signal in each frequency band from an output signal of the processing circuit.

2. The signal transmission circuit according to claim 1, wherein the processing circuit comprises:a plurality of mixers, the mixers mixing the electrical signals with carrier signals corresponding to the frequency bands; anda synthesizer, which synthesizes signals generated by the plurality of mixers to form the output signal of the processing circuit.

3. The signal transmission circuit according to claim 2, wherein the receiving circuit comprises:an amplifying circuit, which amplifies the output signal;an analog-to-digital converter (ADC), which converts the signal amplified by the amplifying circuit into a digital signal;a plurality of multipliers, the multipliers respectively multiplying the plurality of carrier signals with the digital signal outputted by the analog-to-digital converter to extract the electrical signals in the frequency bands from the digital signal; anda plurality of digital filters, the digital filters respectively filtering the signals generated by the corresponding multipliers.

4. The signal transmission circuit according to claim 3, wherein
the signal transmission circuit further comprises:
a numerically controlled oscillator (NCO), which generates a plurality of carrier signals in the form of digital signals; and
a digital-to-analog converter (DAC), which is connected to the numerically controlled oscillator and the plurality of mixers, and converts the plurality of carrier signals in the form of digital signals into a plurality of carrier signals in the form of analog signals, and transmits the plurality of carrier signals in the form of analog signals to the plurality of mixers.

5. The signal transmission circuit according to claim 4, wherein the numerically controlled oscillator is further connected to the plurality of multipliers, and transmits the plurality of carrier signals in the form of digital signals to the plurality of multipliers.

6. The signal transmission circuit according to claim 4, wherein the numerically controlled oscillator, the digital-to-analog converter, and the receiving circuit are integrated into the same chip.

7. The signal transmission circuit according to claim 1, wherein the signal transmission circuit further comprises:an optical communication module, which is connected between the plurality of signal receiving coil elements and the processing circuit, and converts electrical signals received by the plurality of signal receiving coil elements into optical signals, transmits the optical signals to a side of the processing circuit, and converts the optical signals transmitted to the side of the processing circuit into electrical signals to be outputted to the processing circuit.

8. The signal transmission circuit according to claim 7, wherein the optical communication module comprises:a plurality of electro-optical converters, each electro-optical converter being separately connected to a corresponding signal receiving coil element and converting the electrical signal received by the signal receiving coil element into an optical signal;
an optical switch, an input end of the optical switch being connected to the plurality of electro-optical converters, and the optical switch guiding each optical signal to a corresponding transmission channel;
an optical fiber, one end of the optical fiber being connected to an output end of the optical switch, and the optical fiber transmitting the optical signal in the transmission channel;
and a photoelectric converter, which is connected to the other end of the optical fiber, and
converts the optical signal received from the optical fiber into an electrical signal and outputs the electrical signal to the processing circuit.

9. An optical communication module of a magnetic resonance imaging system, characterized by comprising:a plurality of electro-optical converters, each electro-optical converter being separately connected to one of a plurality of signal receiving coil elements of the magnetic resonance imaging system and converting an electrical signal received by the signal receiving coil element into an optical signal;an optical switch, an input end of the optical switch being connected to the plurality of electro-optical converters, and the optical switch guiding each optical signal to a corresponding transmission channel;an optical fiber, one end of the optical fiber being connected to an output end of the optical switch, and the optical fiber transmitting the optical signal in the transmission channel;
and a photoelectric converter, which is connected to the other end of the optical fiber, and
converts the optical signal received from the optical fiber into an electrical signal and outputs the electrical signal to a processing circuit of the magnetic resonance imaging system.

10. A magnetic resonance imaging system, comprising the signal transmission circuit according to claim 1.

11. A magnetic resonance imaging system, comprising the optical communication module according to claim 9.
